(19) United States
(12) Patent Application Publication
(10) Pub. No.: US 2015/0247841 A1
(43) Pub. Date: Sep. 3, 2015

(54) NEUROFURANS-INDICES OF OXIDANT STRESS

(71) Applicant: The Trustees of the University of Pennsylvania, Philadelphia, PA (US)

(72) Inventors: Garret A. Fitzgerald, Wayne, PA (US); John A. Lawson, Penn Valley, PA (US); Wenliang Song, Philadelphia, PA (US)

(21) Appl. No.: 14/643,343

(22) Filed: Mar. 10, 2015

Related U.S. Application Data
(63) Continuation of application No. 12/812,399, filed on Jul. 9, 2010, now abandoned.
(60) Provisional application No. 61/010,852, filed on Jan. 11, 2008.

(51) Int. Cl. G01N 33/53

(52) U.S. Cl. CPC G01N 33/5308; G01N 2800/2821; G01N 2496/00

(57) ABSTRACT

The invention is drawn to a new class of isoeicosanoids that have been identified as products of the oxidation of docosahexaenoic acid (DHA). The invention provides compositions and methods related to the new class of molecules.

### CROSS-REFERENCE TO RELATED APPLICATIONS

This application is a continuation of, and claims priority to, co-pending U.S. patent application Ser. No. 12/812,399, filed Jul. 9, 2010, which is a U.S. national phase application filed under 35 U.S.C. §371 claiming benefit to International Patent Application No. PCT/US09/30632, filed Jan. 9, 2009, which is entitled to priority under 35 U.S.C. §119(e) to U.S. Provisional Patent Application No. 61/010,852, filed Jan. 11, 2008, each of which application is hereby incorporated herein by reference in its entirety.

### STATEMENT REGARDING FEDERALLY SPONSORED RESEARCH OR DEVELOPMENT

This invention was made with government support under grant number PO 1-HL62250 awarded by the National Institutes of Health. The government therefore has certain rights in the invention.

### BACKGROUND OF THE INVENTION

Oxidant stress, characterized by an imbalance between increased exposure to free radicals and antioxidant defenses, is a prominent feature of many acute and chronic diseases. These diseases include neurodegenerative diseases, such as Alzheimer's disease and Parkinson's disease, diabetes, cardiovascular diseases, such as atherosclerosis and hypertension, complications of end-stage renal disease, and pulmonary disorders, such as adult respiratory distress syndrome and chronic obstructive pulmonary disease. Oxidant stress is also thought to play a role in carcinogenesis. Given the prevalence of oxidant stress in disease states and possible role in disease pathogenesis, methods of assessing oxidant stress are of great interest in the medical and research fields. Due to their reactive and thus transitory nature, free radicals are difficult to detect and quantify directly in vivo or in biological samples. Biomarkers of oxidant stress have therefore been sought.

Lipid peroxidation, which results from the oxidative degradation of lipids by free radicals (in vivo, reactive oxygen species (ROS)) is a major manifestation of/contributor to oxidant stress. Thus, identifying biomarkers derived from lipid peroxidation has garnered much attention and effort as biomarkers of oxidant stress.

To date, the most reliable available biomarker of lipid peroxidation in vivo are isoprostanes. Isoprostanes (iPs) are prostaglandin (PG) isomers derived by free radical attack on esterified arachidonic acid (AA) in cell membranes (Morrow et al., 1992, Proc Natl Acad Sci USA. 89(22):10721-5). Once formed in cell membranes, iPs are cleaved, presumably by phospholipases, circulate in plasma and are excreted in urine, where they can be quantified by immunologic methods or by mass spectrometry. Prostaglandin F2a (PGF2a) isomers (F2-iPs) have been measured in urine as indices of in vivo lipid peroxidation (Lawson et al., 1999, J Biol Chem., 274(35):24441-4). F2-iPs have been shown to be elevated in frontal and temporal cortex from Alzheimer's disease patients, as well as in cerebrospinal fluid, making them useful for diagnostic and other purposes (U.S. Pat. No. 6,727,075; Pratico et al., 1998, FASEB J. 12:1777-1783). However, the utility of urinary iPs in this regard has proven controversial.

Compounds analogous to the F₂-iPs are formed from other fatty acid substrates. For example, neuroprostanes (nPs) are iPs derived from the ω-3 fatty acid, docosahexaenoic acid (DHA) (Roberts et al., 1998, J Biol Chem. 273(22):13605-12; U.S. Pat. No. 6,620,800). Given that DHA is more abundant than AA in brain, nPs may prove to be a more attractive biomarker of neurodegeneration than are iPs (Montine et al., 2002, Ann Neurol. 52(2):175-9; U.S. Pat. Publication No. 2003/0211622). Consequently, there is considerable interest in the use of these compounds as indices of progression in neurodegeneration, such as in Alzheimer disease (AD) (Shaw et al., 2007, 6(4):295-303. Epub 2007 Mar. 9). The isofurans (iFs) are a family of free radical-induced peroxidation products of AA (Fessel et al., 2002, Proc Natl Acad Sci USA. 99(26):16713-8). They are even more abundant than iPs in tissues as diverse as kidney and hippocampus in the rat and may offer an adjunctive approach to the assessment of oxidant stress in vivo (Fessel et al., 2002, ibid; Fessel et al., 2003, J Neurochem. 85(3):645-50). Isofurans are formed preferentially under conditions of elevated oxygen tension (Fessel et al., 2002, supra).

### BRIEF SUMMARY OF THE INVENTION

The invention provides a substantially purified isoeicosanoid molecule. The invention further provides a substantially purified composition comprising a plurality of isoeicosanoid molecules. Each isoeicosanoid molecule is characterized by: i) having molecular formula of C₂₂H₃₄O₆; ii) presence of a substituted tetrahydrofuran ring; iii) presence of three hydroxyl groups; iv) presence of four double bonds; v) absence of epoxide groups; and vi) absence of carbonyl groups. The molecule is further characterized by: vii) eluting at m/z 393 in liquid chromatography/mass spectrometry (LC/MS) analysis; and viii) eluting as m/z 609 in gas chromatography/electron capture/negative ionization/mass spectrometry (GC/EC/NI/MS) analysis after pentafluorobenzyl (PFB) ester and trimethylsilyl (TMS) ether derivatization.

The invention further provides a an antibody that specifically binds to an isoeicosanoid molecule, wherein said isoeicosanoid molecule is characterized by: i) having molecular formula of C₂₂H₃₄O₆; ii) presence of a substituted tetrahydrofuran ring; iii) presence of three hydroxyl groups; iv) presence of four double bonds; v) absence of epoxide groups; and vi) absence of carbonyl groups.

Additionally, the invention features a method for detecting a product of lipid peroxidation of docosahexaenoic acid (DHA) in a sample. The method comprises detecting neurofurans (nFs) in said sample. Detection may comprise an immunoassay. In some embodiments, the method further comprises isolating nFs from the sample. In some embodiments, the sample is a biological sample. A biological sample includes brain tissue sample and cerebrospinal fluid. Optionally, the method further comprises assessing the amount of the isolated nFs. Assessing the amount of isolated nFs may comprise liquid chromatography/tandem mass spectroscopy.

The invention also provides a method for measuring lipid peroxidation in a subject mammal. The method comprises assessing the level of a neurofuran in a biological sample from a subject mammal, thereby measuring lipid peroxidation. In some embodiments, the method further comprises comparing the level of a neurofuran in the biological sample to a reference level of the neurofuran. In some embodiments, the reference level is diagnostic for the presence of oxidant stress, such that when the level of neurofuran is the same as or greater than the reference level, the presence of oxidant stress in the subject mammal is indicated. In some embodiments, the reference level is diagnostic for a neurodegenerative disorder. Neurodegenerative disorders include Alzheimer's disease, Parkinson's disease, Huntington's disease and amyotrophic lateral sclerosis (AML). In some embodiments, the reference level is diagnostic for the absence of oxidant stress, such that when the level of neurofuran is the same as or less than the reference level, the absence of oxidant stress in the subject mammal is indicated.

A method for detecting a change in lipid peroxidation in a subject is also provided by the invention. The method comprises the steps of a) assessing a first level of nFs in a biological sample obtained from the subject, and b) assessing a second level of nFs in a biological sample obtained from the subject under a different condition, wherein a difference in the first level compared to the second level is indicative of a change in lipid peroxidation in the subject. The different condition may be one of a different point in time, the presence of a therapeutic agent, the absence of a therapeutic agent and a change in clinical status. In an embodiment, the different condition is the presence of a therapeutic agent and the agent is selected from the group consisting of a DHA-containing agent, a ω-3 fatty acid-containing agent, fish oil, and a combination thereof. In some embodiments, the biological sample is brain tissue, cerebrospinal fluid or plasma.

A method of measuring the level of lipid peroxidation in a mammal suspected of having an oxidant stress syndrome or disease is provided by the invention. The method comprises the steps of assessing the level of a neurofuran present in a first sample of a tissue or body fluid from a mammal; and comparing the level of the neurofuran present in said first sample with the level of the neurofuran present in a second sample of a tissue or body fluid obtained from an otherwise identical mammal which is not afflicted with an oxidant stress syndrome or disease. An elevated level of the neurofuran in the first sample relative to the level of the neurofuran in the second sample is indicative of an elevated level of lipid peroxidation in the mammal, thereby indicating the presence of an oxidant stress syndrome or disease in the mammal. In an embodiment, the tissue is brain tissue, preferably brain cortex tissue. In another embodiment, the body fluid is one of CSF and plasma. In an embodiment, the oxidant stress syndrome or disease is selected from the group consisting of a neurodegenerative disease, a cardiovascular disorder and a pulmonary disorder.

The invention further provides a method of identifying a compound useful for reducing the level of a marker for lipid peroxidation. The method comprises the steps of a) measuring the level of a neurofuran in either a sample of a tissue or body fluid obtained from a first mammal prior to administering the compound or in a sample of a tissue or body fluid obtained from an otherwise identical second mammal which is not to be administered the compound; b) administering the compound to the first mammal; c) thereafter measuring the level of the neurofuran in a tissue or body fluid sample obtained from the first mammal; d) comparing the level of the neurofuran measured in step c) with the level of the neurofuran measured in step a). When the level of the neurofuran measured in step c) is reduced relative to the level of the neurofuran measured in step a), the compound is identified as useful for reducing the level of a marker for lipid peroxidation in a mammal. In an embodiment, the compound is present in an amount effective to reduce the level of a reactive oxygen species in the brain tissue of the mammal.

The invention further comprises kits useful for practicing the methods of the invention. In one aspect, the kit is useful for detecting a neurofuran in a sample. In an embodiment, the kit comprises a neurofuran standard and an instructional material. Optionally, the kit further comprises a solution useful for extracting a neurofuran from a biological sample. In another embodiment, the kit comprises an antibody that specifically binds to a neurofuran and an instructional material. Optionally, the kit further comprises a sample of substantially purified neurofurans. In another aspect, a kit for diagnosing Alzheimer's disease is provided. The kit comprises a) a sample container for carrying a tissue or body fluid sample from a mammal; b) a solution for use in extraction of a neurofuran from the tissue or body fluid sample obtained from the mammal; c) a negative control solution of the neurofuran present at a concentration of about the concentration of said neurofuran present in a tissue or body fluid sample of a mammal which is not afflicted with Alzheimer's disease; d) a positive control solution of the neurofuran present at a concentration of about the concentration of said isoprostane molecular marker in a tissue or body fluid sample of a mammal which is afflicted with Alzheimer's disease; e) an antibody directed against said neurofuran; and f) an instructional material.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts a negative ion mass spectrum chromatogram of an extract of mouse liver after CCl₄ infusion. The spectrum shown is an average of scans across the region of the chromatogram in which iPs (m/z 353), iFs (m/z 369), and nPs (m/z 377) eluted.

FIGS. 2A and 2B depict selected ion monitoring (SIM) chromatograms of an extract of mouse liver after CCl₄ infusion. FIG. 2A depicts chromatograms of peaks eluting at different points. The peaks at m/z 353 are iPs. The peaks at m/z 377 are nPs. The peaks at m/z 369 are iFs. The peaks at m/z 393 are novel. FIG. 2B depicts chromatograms of the FIG. 2A peaks after PFB ester, TMS ether derivatization and SIM analysis by gas chromatography/electron capture/negative ionization mass spectrometry (GC/EC/NI/MS).

FIG. 3 depicts an LC/MS/MS comparison of in vivo and in vitro products of DHA oxidation. Left: In vitro. Right: In vivo.

FIG. 4 is a series of GC/MS chromatograms before and after formation of [²H₉]TMS ether derivatives to assess the presence of hydroxyl groups in the ion at m/z 393.

FIGS. 5A and 5b are a series of chromatograms relating to the characterization of the ion at m/z 393. FIG. 5A depicts a series of GC/MS chromatograms before and after catalytic hydrogenation to assess the presence of double bonds in the ion at m/z 393. FIG. 5B depicts a series of SIM chromatograms before treatment with HCl or methoxyamine HCl (top), after treatment with HCl (middle) and after treatment with methyoxyamine HCl (bottom).

FIG. 6 depicts the cyclic peroxide cleavage pathway of nF formation.

FIG. 7 depicts the epoxide hydrolysis pathway of nF formation.

FIG. 8 depicts an LC/MS/MS product ion scans of m/z 393 of products of DHA oxidation. Left: in vitro DHA oxidation. Right: in vivo DHA oxidation (from an extract of mouse liver after CCl₄).

FIGS. 9A and 9B relate to predicted product ion formation based on the proposed nF structures. FIG. 9A tabulates the main MS/MS fragments for each of 16 proposed nF structures. FIG. 9B depicts MS/MS fragmentation sites to yield the fragments in the table in FIG. 9A.

FIG. 10 is a series of chromatograms obtained from LC/MS/MS analysis of the products of in vitro DHA oxidation. SRM analysis of some predicted transitions of m/z 393 from the in vitro oxidation of DHA is shown.

FIG. 11 is a graph depicting the time course of nF formation during in vitro oxidation of DHA with 2,2′-azobis(2-amidinopropane)hydrochloride (AAPH). (SRM analysis of m/z 393→193 and 377→101).

FIGS. 12A and 12B are graphs related to characterizing nFs. FIG. 12A is a graph depicting the dose response relationship between nF and nP formation and 2,2′-azobis(2-amidinopropane)hydrochloride (AAPH) concentration during in vitro oxidation of DHA. (SRM analysis of m/z 393→193 and 377→101). FIG. 12B is a graph depicting the formation nFs under various oxygen tension. (SIM analysis of m/z 393, n=8).

FIG. 13 is a graph depicting the formation of nFs in liver following intraperitoneal injection of CCl₄ (4 g/kg). Time after injection is indicated on the x axis. (SRM analysis of m/z 393→193 and 377→101).

FIGS. 14A and 14B are graphs relating to the relative amounts of iPs, nPs and nFs in the brain cortex of Tg 2576 transgenic mice and non-transgenic littermates (controls). (SRM analysis of m/z 393→193 and 377→101) FIG. 14A depicts data for AD brain cortex (from Tg 2576 transgenic mice) compared to control brain cortex (from non-transgenic litter mates). *p<0.05; **p<0.01 FIG. 14B depicts data for AD cerebellum compared to control brain cortex.

FIG. 15 depicts nF levels in p47 phox knockout mice compared to wild type controls. (SRM analysis of m/z 393→193 and 377→101). *p<0.05; n=10.

### DETAILED DESCRIPTION OF THE INVENTION

The invention arises from the discovery of a novel class of isofuran (iF)-like compounds, named neurofurans (nFs), that are formed in vivo and in vitro from the free radical-initiated peroxidation of docosahexaenoic acid (DHA). It has further been discovered that nFs are highly abundant in brain. Notably, nFs are more abundant than iPs in the brain.

Consequently, the present application features substantially purified neurofurans and derivatives thereof, compositions comprising substantially purified neurofurans, as well as methods for detecting the same, for instance, in a biological specimen. The method is useful in many applications, including but not limited to, research, diagnostic and clinical.

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

It is understood that any and all whole or partial integers between any ranges set forth herein are included herein.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

“Instructional material,” as that term is used herein, includes a publication, a recording, a diagram, or any other medium of expression which can be used to communicate the usefulness of the composition in the kit in the practice of a method of the invention. The instructional material of the kit may, for example, be affixed to a container that contains the composition of the invention or be shipped together with a container which contains the compound and/or composition. Alternatively, the instructional material may be shipped separately from the container with the intention that the recipient uses the instructional material and the composition cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

By the term “specifically binds,” as used herein with respect to an antibody, is meant an antibody which recognizes an specific antigen, but does not substantially recognize or bind other molecules in a sample. For instance, in a sample containing neurofurans, an antibody that specifically binds to neurofurans recognizes and binds to neurofurans but does not substantially recognize or bind to other molecules in the sample.

As used herein, the term “oxidant stress” means the consequences of free radical dependent damage to proteins, DNA and/or lipid without regard to the specific radical involved or the relative preponderance of the targets. “Oxidant stress” implies radical generation in excess of that which can be quenched (i.e., coped with) by the endogenous antioxidant defenses of a mammal, and implies tissue or organ dysfunction in the mammal, and is thus a potential mechanism of disease.

As used herein, the term “oxidant stress syndrome or disease” means any disease or syndrome either caused by oxidant stress or of which oxidant stress is a symptom. For example, a neurodegenerative oxidant stress disease is a neurodegenerative disease which is either caused by oxidant stress, or of which oxidant stress is a symptom.

As used herein, the term “lipid peroxidation” means the consequence of free radical damage to lipids.

As used herein, “to diagnose” means to determine or distinguish the nature of a problem or an illness through a diagnostic analysis. Diagnostic analysis generally involves performing one or more tests that directly or indirectly measure a sign, substance, response, or tissue change that is either an absolute or reasonable surrogate predictor of a disease or disease agent. A single diagnostic test typically provides a likely diagnosis, not a definitive diagnosis. A diagnosis can encompass diagnosis of an elevated risk for a disorder or disease.

As used herein, an “elevated risk” for a disorder or disease refers to an increase in the likelihood or possibility of developing that disorder or disease. This risk can be assessed relative to a patient's own risk, or with respect to a population that does not have clinical evidence of the disorder or disease and/or is not at risk for the disorder or disease. The population may be representative of the patient with regard to particular parameters, for instance, approximate age, age group, gender, other clinical conditions and the like.

As used herein, a “reference level” refers to a level of one or more neurofurans obtained from one mammal or more than one mammal and which is indicative of a condition in the one or more mammals. For instance, a reference level obtained from one or more mammals with clinical evidence of oxidant stress is considered diagnostic for the presence of oxidant stress. Optionally, the one or more mammals used in obtaining the reference level are diagnosed with the same disease characterized by the presence of oxidant stress; the reference level is therefore diagnostic for an elevated risk of or likely presence of the disease.

As used herein, a level is “the same as a reference level” if the level is within a statistically significant range of the reference level. The skilled artisan is familiar with methods in the art for determining such a reference level and a statistically significant range. In some embodiments, “statistically significant range” refers to a range established by a standard deviation around an arithmetic mean. In other embodiments, a “statistically significant range” refers to a range defined as plus or minus two standard errors around the arithmetic mean.

As used herein, “neurofuran” refers to an isoeicosanoid that has the following features: having the molecular formula C₂₂H₃₄O₆, a substituted tetrahydrofuran ring, three hydroxyl groups, four double bonds, and no epoxide or carbonyl groups. Neurofurans are identified herein as the product of free radical-catalyzed oxidation of DHA.

As used here, a “neurofuran metabolite” refers to a byproduct of neurofuran metabolism in an animal, preferably in a mammal.

As used herein, the term “substantially purified” or “substantially pure” means a compound, e.g., a protein or a lipid, or population of isomers, e.g., regioisomers and/or racemic diastereomers, which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of lipids by low, medium or high pressure liquid chromatography, thin layer chromatography, gas spectrometry, or mass spectrometry. A compound, e.g., a lipid, is also substantially purified when it is essentially free of naturally associated components or when it is separated from the native contaminants which accompany it in its natural state. Included within the meaning of the term “substantially pure” as used herein is a compound, such as a lipid, which is homogeneously pure, for example, where at least 95% of the total lipid (by volume, by wet or dry weight, or by mole percent or mole fraction) in a sample is the lipid of interest.

It is understood that any and all whole and partial integers between the ranges set forth here are included herein. With respect to any figure or numerical range for a given characteristic, a figure or a parameter from one range may be combined with another figure or a parameter from a different range for the same characteristic to generate a numerical range.

The following abbreviations are used: arachidonic acid (AA); 2,2′-azobis(2-amidinopropane)hydrochloride (AAPH); Alzheimer's disease (AD); docosahexaenoic acid (DHA); electron capture (EC); gas chromatography (GC); isoprostanes (iPs); isofurans (iFs); liquid chromatography (LC); mass spectrometry (MS); negative ionization (NI); neurofurans (nFs); neuroprostanes (nPs); prostaglandin (PG); selective ion monitoring (SIM); selective reaction monitoring (SRM); solid phase extraction (SPE); trimethylsilyl (TMS).

### Description

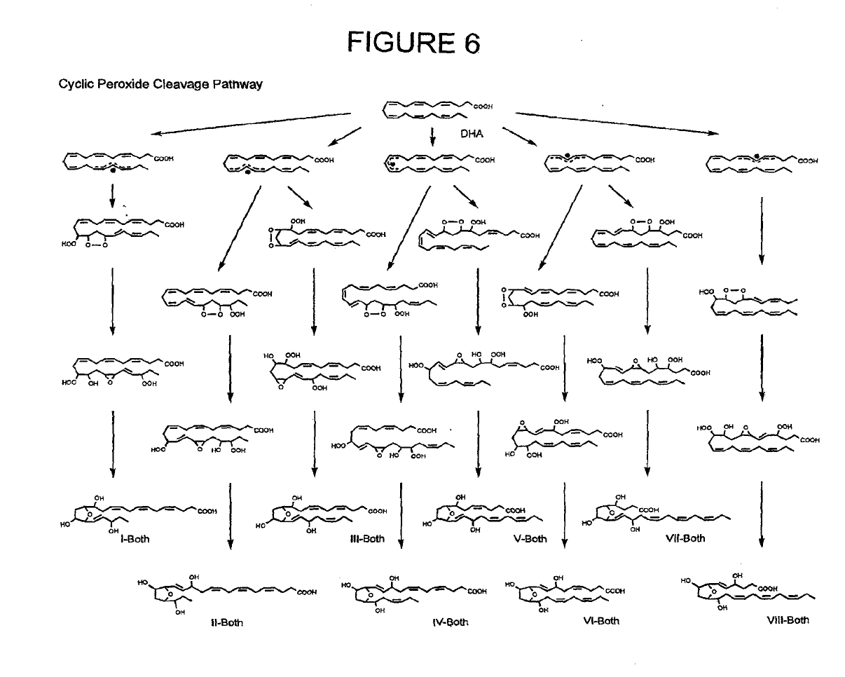

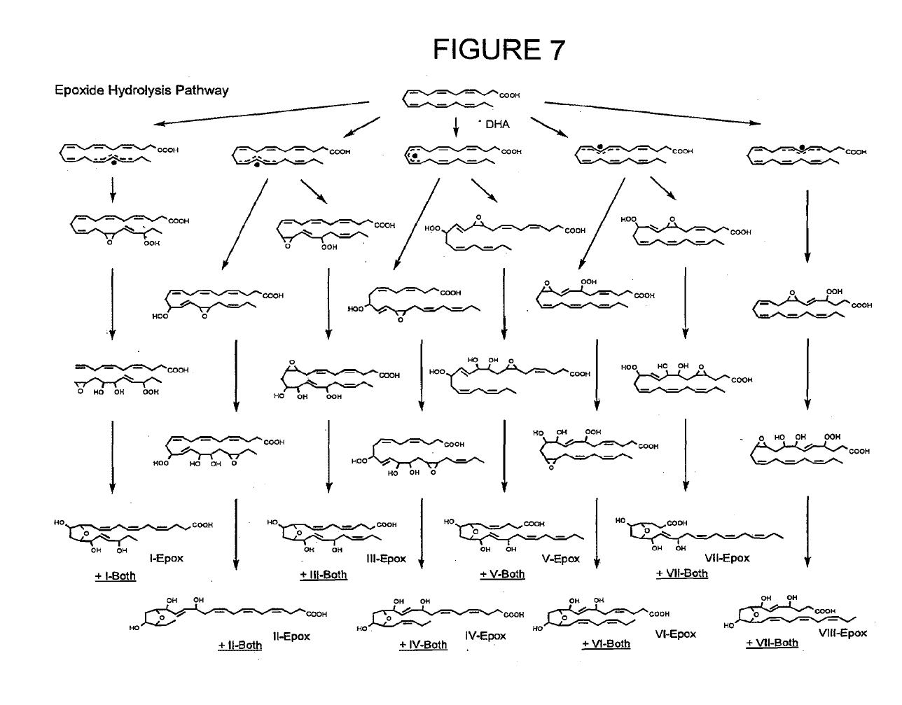

The invention provides a substantially purified isoeicosanoid molecule. The invention further provides a substantially purified composition comprising a plurality of isoeicosanoid molecules, wherein the plurality is of two or more isomeric molecules. An isoeicosanoid molecule is,characterized by the following: i) having molecular formula of C₂₂H₃₄O₆; ii) presence of a substituted tetrahydrofuran ring; iii) presence of three hydroxyl groups; iv) presence of four double bonds; v) absence of epoxide groups; and vi) absence of carbonyl groups. An isoeicosanoid molecule is further characterized by: vii) eluting at m/z 393 in LC/MS analysis; and viii) eluting as m/z 609 in GC/EC/NI/MS analysis after PFB ester and TMS ether derivatization. The isoeicosanoid molecules have been discovered herein to be the product of free radical-initiated peroxidation of docosahexaenoic acid (DHA) either in vivo or in vitro. Proposed pathways for the production of isoeicosanoid molecule are shown in FIGS. 6 and 7. These two pathways predict 16 distinct regioisomers, each comprised of 32 racemic diastereomers for a total of 512 possible compounds. Thus, the class of isoeicosanoid molecules of the invention comprises a total of 512 possible isomers. Isoeicosanoid molecules are referred to herein as “neurofurans” (nFs).

Neurofurans are chemically stable and are abundant, particularly in tissues enriched in DHA, such as the brain. As demonstrated herein, and in contrast to isofurans, formation of nFs in vivo is not detectably favored by increasing oxygen tension. nFs are suitable analytes for oxidant stress in the brain, enabling quantitative and robust measurement of lipid peroxidation in the brain. Accordingly, nFs are useful in research applications, as well as for medical applications. Metabolites of nFs are expected to be similarly abundant, particularly in plasma and in urine. Accordingly, the invention contemplates use of one or more nF metabolites in any method described herein for nFs. The invention further provides a composition comprising nFs and at least one other component, and an antibody that specifically binds nFs. The antibody may be polyclonal or monoclonal. Components useful in the composition include, but are not limited to, inert diluents; excipients; carriers; aqueous vehicles and solvents; oily vehicles and solvents; suspending agents; dispersing or wetting agents and the like.

The invention further encompasses derivatives of nFs. Non-limiting examples of nF derivatives include TMS ether derivatives and derivatives obtained from PFB ester and TMS ether derivatization. The skilled artisan will recognize that other derivatives exist, and these are encompassed by the invention.

The invention further encompasses metabolites of nFs and derivatives thereof. nF metabolites are expected to be detectable in biological samples and may be isolated and their level assessed by the same or similar methods used for isolating and assessing nFs.

The nFs of the invention may be obtained by purification from a biological sample or may be synthesized chemically using standard techniques known in the art, such as treating DHA with an oxidant. An exemplary oxidant for this purpose is 2,2′-azobis(2-amidinopropane)hydrochloride (AAPH).

“Biological sample” as used herein refers to a tissue sample or body fluid. Exemplary tissues are tissues enriched in DHA. Tissues useful as tissue samples in the invention include brain tissue and liver tissue. Measurements made using a sample of body fluid can be made in any type of body fluid. Exemplary body fluids include, but are not limited to, cerebrospinal fluid (CSF), blood and blood components, e.g., serum or plasma.

The biological sample may be obtained from any animal that has endogenous DHA. Non-limiting examples of animals from which a biological sample may be obtained are mammals, such as humans, non-human primates, cattle, horses, dogs, sheep, goats, mice, rats and pigs. Preferably, the animal is a human. Methods of obtaining biological samples are well known in the art and include, but are not limited to, biopsy, surgical sample, lumbar puncture, venous puncture and the like.

nFs may be purified from the biological sample using any method known in the art. Useful isolation methods include, by way of example, and not by limitation, purification methods such as solvent extractions, solid phase extractions, chromatographic methods, centrifugation and sedimentation methods, among others. Chromatographic methods include affinity chromatography using an antibody that specifically binds to nFs, gas chromatograph methods, mass spectrometry methods, thin-layer chromatography and low, medium and high pressure liquid chromatography (HPLC). An exemplary purification method using liquid chromatography/tandem mass spectrometry (LC/MS/MS) is described in the Examples.

Another preferred method of isolating nFs is as follows. Briefly, the nFs are isolated by first, in the case of a tissue sample, homogenizing the tissue sample, mechanically or enzymatically or both. In the case of a body fluid sample, no homogenization step is necessary. Total lipids are then extracted from the sample using a modified Folch procedure. Samples are suspended ice-cold chloroform/methanol (2:1, v/v), with an oxidation suppressant. An exemplary oxidation suppressant is butylated hydroxytoluene, at 0.005% (w/v). Lipid extracts are then mixed with a salt solution, e.g. 0.9% (w/v) NaCl, and centrifuged. The organic phase, which contains the extracted lipids, is dried under nitrogen.

Assessing the level of nFs may be achieved using techniques and methods known to the skilled artisan for assessing, measuring, assaying or quantifying an isoprostane molecule. Such methods are described, for example, in Lawson et al. (1999, J. Biol. Chem., 374(35) 24441-24444). These methods include, by way of example, and not by limitation, quantitative and semi-quantitative methods such as chromatographic methods including thin layer chromatography, low, medium, and high pressure liquid chromatography methods, mass spectrometry methods, gas chromatography methods, gas chromatography/mass spectrometry methods, and immunological methods. Preferably, the assay is a quantitative assay. The level of nFs is quantified based on the assay results using, for example, peak area or peak height ratios. An example of assessing the level of nFs in a biological sample using LC/MS/MS is described herein in the Examples. Where appropriate, levels of nFs in a sample may be normalized, for example, to another component typically found in the same sample type. The skilled artisan is familiar with how to identify such a component. Preferably, the other component is known to be generally constant in a biological sample or relatively constant in a known oxidant stress disease state.

Assessing the level of nFs in a biological sample is preferably accomplished using LC/MS/MS as described elsewhere herein. However, the skilled artisan may use any quantitative method of assessing the level of a prostaglandin.

Another method for assessing the level of nFs isolated using the above-described total lipid extraction method includes the following steps: the sample which contains the nFs is spiked with a known amount of a synthetic homologous internal standard or an internal standard, such as [²H₄]8,12-iso-iPF_{2α}-VI. The samples are then subjected to solid phase extraction, derivatized, and purified using thin layer chromatography. After thin layer chromatography, each sample is analyzed for nF content using gas chromatography-mass spectrometry, and quantification is performed using peak area or peak height ratios.

Yet another method for assessing the level of neurofurans is the use of an immunoassay which employs an antibody that specifically binds neurofurans. Preferably, the anti-neurofuran antibody is monoclonal. While any known immunoassay can be utilized to test for nFs, the preferred immunoassay is an ELISA. Methods for the preparation and purification of antibodies are known in the art, and are described, for example, in Harlow et al., 1988, Antibodies: A Laboratory Manual, Cold Spring Harbor, N.Y. Antibodies of the present invention may exist in a variety of forms including, for example, polyclonal antibodies, monoclonal antibodies, intracellular antibodies (“intrabodies”), Fv, Fab and F(ab)₂, as well as single chain antibodies (scFv), heavy chain antibodies, such as camelid antibodies, and humanized antibodies.

Immunoassays useful in the present invention include for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1988, supra; Harlow et al., 1999, Using Antibodies: A Laboratory Manual, Cold Spring Harbor Laboratory Press, NY.

A method of detecting or measuring lipid peroxidation is provided by the invention. The method comprises detecting nFs or assessing the level of nFs in a sample. Any detection method or assessment method known in the art may be used, including those described elsewhere herein. Non-limiting examples of such methods include LC/MS/MS and immunoassays. Such a method is useful in many different applications. In one embodiment, the sample is a biological sample. A kit useful for the practice of this method is also provided. The kit comprises a neurofuran standard and an instructional material for using the kit to detect nFs in a sample. The kit optionally comprises a container for the sample. In one embodiment, the kit further comprises a solution useful for extracting a neurofuran from a biological sample. A kit comprising an antibody that specifically binds to neurofurans and an instructional material is also provided. In one embodiment, the kit further comprises substantially purified neurofurans, which are useful as a standard or positive control.

Neurofurans are shown herein to be free-radical peroxidation products of DHA in vivo. Thus, the invention further provides a method for measuring lipid peroxidation in a subject mammal. The method includes the step of assessing the level of a neurofuran in a biological sample from said subject mammal, thereby measuring lipid peroxidation. In one embodiment, the method further comprises the step of comparing the level of neurofuran in the biological sample to a reference level of the neurofuran. Where the reference level is diagnostic for the presence of oxidant stress, if the level of neurofuran in the biological sample is the same as or greater than the reference level, the presence of oxidant stress is indicated in the subject mammal. Where the reference level reflects the absence of oxidant stress, if the level of neurofuran in the biological sample is the same as or less than the reference level, the presence of oxidant stress is indicated in the subject mammal.

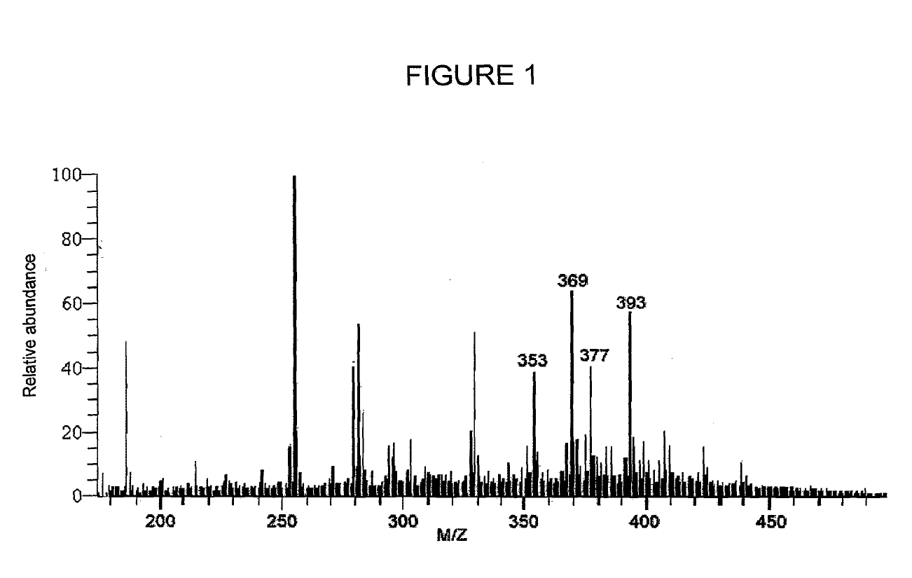

As the skilled artisan is aware, the accuracy of a diagnostic test is commonly measured by its sensitivity and specificity. The skilled artisan is well aware that distributions of data from healthy and diseased persons almost always overlap (see, for instance, page 6 of Sacher et al., Widmann's Clinical Interpretation of Laboratory Tests, “Principles of Interpretation of Laboratory Tests”, F.A. Davis Company, Philadelphia, Pa., 2000, pp. 3-27). As discussed in Sacher et al., the region of overlap concerns “false positives” in the healthy group and “false negatives” in the diseased group. Sensitivity and specificity, which are calculated based on a threshold value separating values identified as “healthy” and values identified as “diseased” (Ibid, pp. 5-6, FIG. 1-1 and text), describe the frequency of such false negatives and false positives, respectively, for a diagnostic dataset. While the ideal diagnostic test would have both specificity and sensitivity of 100%, that is, a test with no false positives and no false negatives, this high standard cannot generally be met (Ibid, p. 6, final paragraph). That is, the typical diagnostic test is for a likely diagnosis, not a definitive diagnosis. In some embodiments, the reference level is a threshold value as discussed in Sacher et al. (ibid). In some embodiments herein, the reference level is a threshold value selected for a high specificity. In other embodiments, the reference level is a threshold value selected for a high sensitivity.

The invention further provides a method for detecting a change in lipid peroxidation in a subject. In this method, a first level of nFs in a biological sample obtained from a subject is assessed and a second level is assessed under a different condition. A difference in said first level to said second level is indicative of a change in lipid peroxidation in said subject. Preferably, the difference is statistically significant with a p<0.05, and more preferably p<0.05 and more preferably still, p<0.01. Preferably the first and second levels are measured in the same type of biological sample. Preferably the first and second levels are measured using the same method, for instance, LC/MS/MS. Different conditions include, but are not limited to, a different point in time, the presence or withdrawal (absence) of a therapeutic agent, and a change in the clinical status of the subject. In one embodiment, the method is used to monitor the effect of a therapeutic agent for increasing DHA in vivo. Such therapeutic agents include, but are not limited to, a DHA-containing agent, a ω-3 fatty acid agent, fish oil or a combination thereof.

The invention further provides a method of measuring the level of lipid peroxidation in a mammal suspected of having an oxidant stress syndrome or disease. The method includes the steps of a) obtaining a first sample of a tissue or body fluid from said mammal; b) assessing the level of a neurofuran present in said first sample; and c) comparing the level of said neurofuran present in said first sample with the level of said neurofuran present in a second sample of a tissue or body fluid obtained from an otherwise identical mammal which is not afflicted with an oxidant stress syndrome or disease, wherein an elevated level of said neurofuran in said first sample relative to the level of said neurofuran in said second sample, is indicative of an elevated level of lipid peroxidation in said mammal, thereby indicating the presence of an oxidant stress syndrome or disease in said mammal. The oxidant stress syndrome or disease may be any disorder or disease characterized by oxidant stress. In one embodiment, it is selected from the group consisting of a neurodegenerative disease, a cardiovascular disease and a pulmonary disease. Exemplary neurodegenerative diseases include Alzheimer's disease, Parkinson's disease, Huntington's disease and AML. Exemplary cardiovascular diseases include atherosclerosis and hypertension. Pulmonary diseases include adult respiratory distress syndrome and chronic obstructive pulmonary disease. Other diseases associated with oxidant stress include diabetes, including complications of diabetes, complications in end-stage renal disease and Downs syndrome.

The method of the invention can be performed either on a subject which manifests a symptom or symptoms of oxidant stress or disease characterized by the presence of oxidant stress, or on a subject which does not manifest a symptom or symptoms of oxidant stress or disease characterized by the presence of oxidant stress. Furthermore, the methods of the invention may be performed on the subject at any stage in the progression of a disease characterized by oxidant distress. Additionally, the methods of the invention may be performed on a subject suspected of having a change, either an increase or a decrease, in oxidant stress.

As shown herein, the peroxidation products of DHA are more abundant in the brain of a mouse model of AD than those in the brain of a wild type control mouse. The prior art has demonstrated greater free radical damage in DHA- than AA-containing compartments in diseased regions of AD brain, and suggest diminished reducing capacity in DHA-containing compartments (Reich et al., 2001, Am J Pathol. 158(1):293-7; Nourooz-Zadeh et al., 199, J Neurochem. 72: 734-740; Montine et al., 2004, Chem Phys Lipids 128(1-2):117-24). Furthermore, it is known that the brains of AD patients are relatively deficient in DHA in the gray matter of the frontal lobe and hippocampus (Söderberg et al., 1991, Lipids 26: 421-5). Thus, quantification of DHA oxidation products, specifically nFs, is contemplated to be a sensitive indicator of oxidant stress in brain and is further contemplated to be, advantageously, more sensitive an indicator than measurement of either iPs or iFs. More specifically, assessment of nFs is contemplated to be useful in diagnosing possible or probable Alzheimer's disease in a human subject. Since isoprostanes are found in both CSF and blood, it is further contemplated that the sheer abundance of nFs in the brain will lead to a similarly high abundance of nFs in CSF and blood, especially plasma.

Accordingly, in one embodiment of the method of measuring the level of lipid peroxidation in a mammal suspected of having an oxidant stress syndrome or disease, the oxidant stress disease is Alzheimer's disease. A kit useful for diagnosing Alzheimer's disease in a mammal is also included in the invention. The kit comprises: a) a sample container for carrying a tissue or body fluid sample from a mammal; b) a solution for use in extraction of a neurofuran from the tissue or body fluid sample obtained from the mammal; c) a negative control solution of said neurofuran present at a concentration of about the concentration of said neurofuran present in a tissue or body fluid sample of a mammal which is not afflicted with Alzheimer's disease; d) a positive control solution of the neurofuran present at a concentration of about the concentration of the neurofuran in a tissue or body fluid sample of a mammal which is afflicted with Alzheimer's disease; e) an antibody directed against said neurofuran; and f) an instructional material.

The invention also provides a method of identifying a compound useful for reducing the level of a marker for lipid peroxidation. This method includes the steps of: a) measuring the level of a neurofuran in either a sample of a tissue or body fluid obtained from a first mammal prior to administering the compound, or, in a sample of a tissue or body fluid obtained from an otherwise identical second mammal which is not to be administered the compound; b) administering said compound to the first mammal; c) thereafter measuring the level of the neurofuran in a tissue or body fluid sample obtained from the first mammal; and d) comparing the level of the neurofuran measured in c) with the level of the neurofuran measured in a). When the level of the neurofuran measured in c) is reduced relative to the level of the neurofuran measured in a), a compound useful for reducing the level of a marker for lipid peroxidation in a mammal is identified. Preferably, the reduced level is a level which is from about 60% to about 100% lower than the level of the neurofuran in the sample obtained from the untreated mammal (the otherwise identical second mammal) or in the first mammal prior to administration of the compound. In a preferred embodiment, the first and second mammal are animal models for a neurodegenerative disease such as Alzheimer's disease.

In the method of identifying a compound, the compound can be any compound, including but not limited to an polypeptide, a nucleic acid, a small molecule and combinations thereof. Compounds identified as reducing the level of neurofurans in a mammal are candidates as therapeutic agents for an oxidant stress syndrome or disease.

For any diagnostic method of the invention as well as the method of identifying a compound, an embodiment is contemplated in which the method comprises assessing the level of both a neurofuran and a neuroprostane. The combined analysis of nFs and nPs is contemplated to reflect lipid oxidation in DHA-rich tissues even more comprehensively than either alone.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods used in the experiments presented in the Experimental Examples below are now described.

In Vivo Oxidation—Analysis of Liver from CCl₄-Treated Mice: Treatment of mice with CCl₄ was used to induce oxidant injury to the liver as previously described (Kadiiska et al, 2005, Radic Biol Med. 38(6):711-8). Three month-old C57/BL6 male mice were fasted overnight and then administered intraperitoneal (i.p.) injections of CCl₄ (4 g/kg body weight). CCl₄ was mixed with canola oil (1:1 by volume). Mice were anesthetized with CO₂ prior to sacrifice at 0, 1, 2.5, 7.5, or 20 hours after CCl₄ administration, their livers were removed and rapidly frozen in liquid nitrogen prior to storage at −80° C. Total lipids were extracted using a modified Folch procedure (Pratico et al., 1997, J Clin Invest. 100(8):2028-34. Erratum in: J Clin Invest 1997 Nov. 15; 100(10):2637). Briefly, frozen samples (0.1-0.5 g) were suspended in 5 mL ice-cold chloroform:methanol (2:1, v/v) with 0.005% (w/v) butylated hydroxytoluene to suppress oxidation. Samples were homogenized using a TissueLyzer™ (Qiagen, Valencia, Calif.). The lipid extracts were mixed vigorously with 2.0 mL NaCl (0.9%, w/v), and the phases separated by centrifugation. After the upper phase was decanted, samples were transferred to clean tubes, and the residual organic solvent was removed under a stream of nitrogen. Total lipids were dissolved in 0.5 mL methanol and stored at −80° C. Lipid extracts were then saponified by adding 0.5 mL of 2.7 N KOH in 0.5 mL methanol. The mixture was then sonicated and mixed vigorously until thoroughly suspended, then heated at 37° C. for 30 minutes. The pH was adjusted to 3.0 with 1.2 mL 1N HCl. Next, 1.0 ng of [²H₄]8,12-iso-iPF_{2α}-VI was added as an internal standard. The samples were purified by solid phase extraction (SPE) using Strata™-X cartridges (Phenomenex, Torrance, Calif.). Purified lipid extracts were analyzed by liquid chromatography/tandem mass spectrometry (LC/MS/MS).

In Vitro Oxidation of DHA: Docosahexaenoic acid (DHA), obtained from Cayman Chemical Co. (Ann Arbor, Mich.) was dissolved in ethanol and added to 5 mL PBS, pH 7.4, to a final concentration of 20 mM. 2,2′-azobis(2-amidinopropane)hydrochloride (AAPH; Sigma, St. Louis, Mo.) was used as an oxidant as described (Fessel et al., 2002, Proc Natl Acad Sci USA. 99(26):16713-8). Reactions were allowed to proceed at 37° C., and aliquots were removed for analysis at 0, 2, 4, 8, 12, 24, and 30 hours. Reactions were stopped by immersing the tube in an ice water bath. The aliquots were diluted by a factor of 100 with water. 1.5 ng of [²H₄]8,12-iso-iPF_{2α}-VI was added as internal standard to 10 μL aliquots of the diluted samples. They were then acidified to pH<3 with formic acid and extracted with ethyl acetate. The samples were then purified by SPE before LC/MS/MS analysis. AAPH concentrations of 0.01, 0.1, 1, 10, and 100 mM were used to examine dose dependent product formation. Three independent experiments were done for the time course and dose dependent formation experiments.

MS Analyses: Unknown compounds were characterized by stable isotope dilution gas chromatography/electron capture/negative ionization (GC/EC/NI)/MS as the pentafluorobenzyl (PFB) ester, trimethylsilyl (TMS) ether derivative as described (Fessel et al., 2002, Proc Natl Acad Sci USA. 99(26):16713-8). [²H₉]TMS ether derivatization was used to determine the number of the hydroxyl groups. Catalytic hydrogenation was used to reveal the number of double bonds. Compounds were also subjected to methoxime (MO) derivatization conditions to exclude the presence of ketone or aldehyde groups. Exposure to HCl excluded the possibility of epoxides. Fragmentation patterns of the unknown compounds were analyzed by electrospray (ES) MS/MS in the negative ion mode. [²H₄]8,12-iso-iPF_{2α}-VI was used as an internal standard for quantitation.

Analysis of nFs in Vivo:

(i) P47phox Knockout Mice: Mice lacking with the P47phox component of NADPH oxidase (kindly provided by Steven M. Holland, M.D., NIAID) possess an impaired ability to generate superoxide (Lavigne et al., 2001, 15(2):285-7. Epub 2000 Dec. 8). Ten knockout mice (5 male and 5 female) and ten wild type controls (5 male and 5 female) were anesthetized prior to sacrifice and removal of the brain at the age of 9 months. The brains were rapidly frozen in liquid nitrogen prior to placement at −80° C. Total lipids were extracted from the brains using a modified Folch procedure. Purified lipid extracts were analyzed by LC/MS/MS. [²H₄]8,12-iso-iPF_{2α}-VI was added as an internal standard.

(ii) Tg 2576 transgenic mouse model of AD: Eight female Tg 2576 transgenic mice and 8 female littermate controls were anesthetized prior to sacrifice and removal of their brains at the age of 15 months. The brains were rapidly frozen in liquid nitrogen prior to placement at −80° C. Total lipids were extracted and purified as described above. Purified lipid extracts were analyzed by LC/MS/MS. [²H₄]8,12-iso-iPF_{2α}-VI was added as internal standard.

The results of the experiments are now described.

### Experimental Example 1

### Discovery of the Neurofurans (nFs)

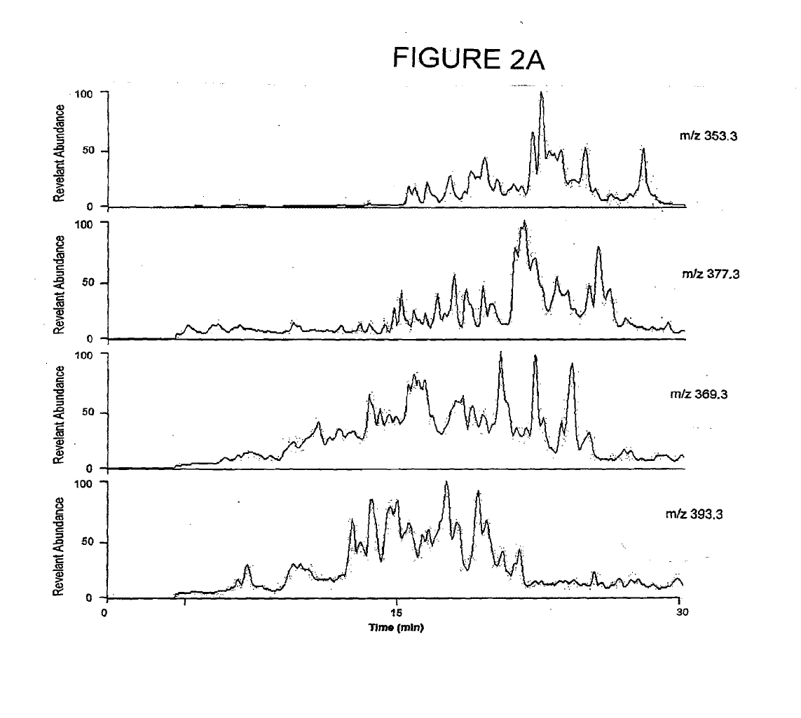

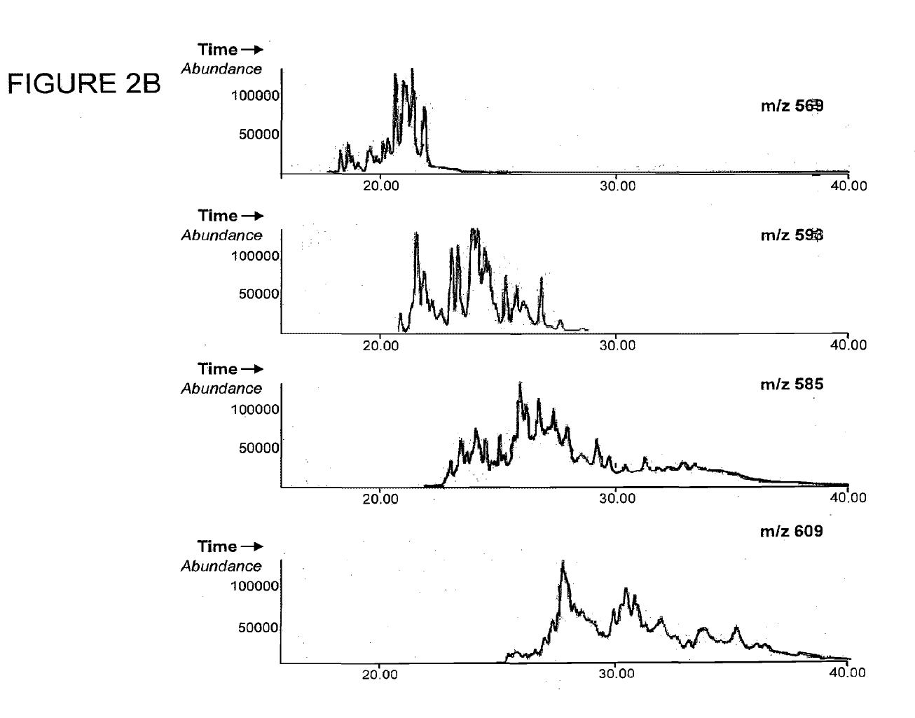

Purified lipid extracts from mouse liver were analyzed by LC/MS/MS after treatment with CCl₄. During the analysis, four ions detected near the region of the chromatogram in which the iPs eluted attracted attention. The ions were m/z 353 (F₂-iPs), m/z 369 (iFs), m/z 377 (nPs) and a peak at m/z 393 (FIG. 1). Selected ion monitoring (SIM) analysis of m/z 393 revealed a region of incompletely resolved chromatographic peaks that eluted slightly earlier than the iPs, nPs and iFs in a reverse phase LC gradient (FIG. 2A). This ion is 16 Da higher than the quasi-molecular ion of nPs (m/z 377) and 24 Da higher than that of iFs (m/z 369). Following GC/EC/NI/MS analysis after PFB ester and TMS ether derivatization, a series of chromatographic peaks was detected at m/z 609, which is again 16 Da higher than nPs (m/z 593) and 24 Da higher than iFs (m/z 585) (FIG. 2B).

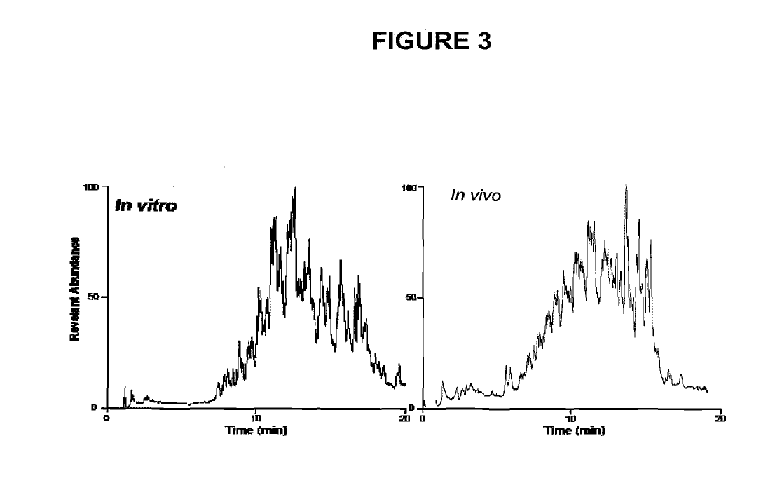

Given these chromatographic, mass spectrometric, and functional similarities, it was surmised that the unknown compounds may share structural similarities with the known products of AA and DHA oxidation, for example, iF-like compounds derived from DHA. To test this hypothesis, an experiment was performed to determine whether these compounds could be generated by oxidation of DHA. After free radical-initiated oxidation of DHA, total lipids were extracted with ethyl acetate and the samples were then purified and analyzed by LC/MS/MS. SIM analysis at m/z 393 revealed similar profiles when the products of in vitro oxidation of DHA were compared with an extract of mouse liver after CCl₄ infusion (FIG. 3).

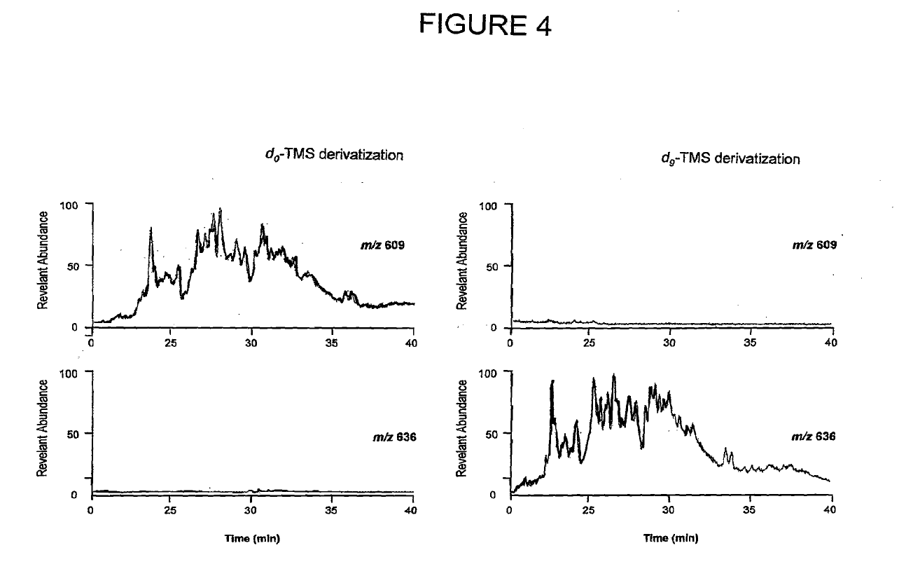

GC/EC/NI/MS analysis of the compounds as the [²H₉]TMS ether derivatives resulted in a mass increase of 27 Da while retaining a similar chromatographic pattern. Specifically, no significant peaks are detectable in the products of in vitro oxidation of DHA at m/z 636 after formation of the PFB ester, TMS derivative. However, following formation of the [²H₉]TMS ether derivative, intense peaks appear at m/z 636 while no peaks are evident at m/z 609, indicating the presence of three hydroxyl groups (FIG. 4). Retention of an almost identical chromatographic pattern with little residual at m/z 609 supports the contention that all of these compounds have three hydroxyl groups.

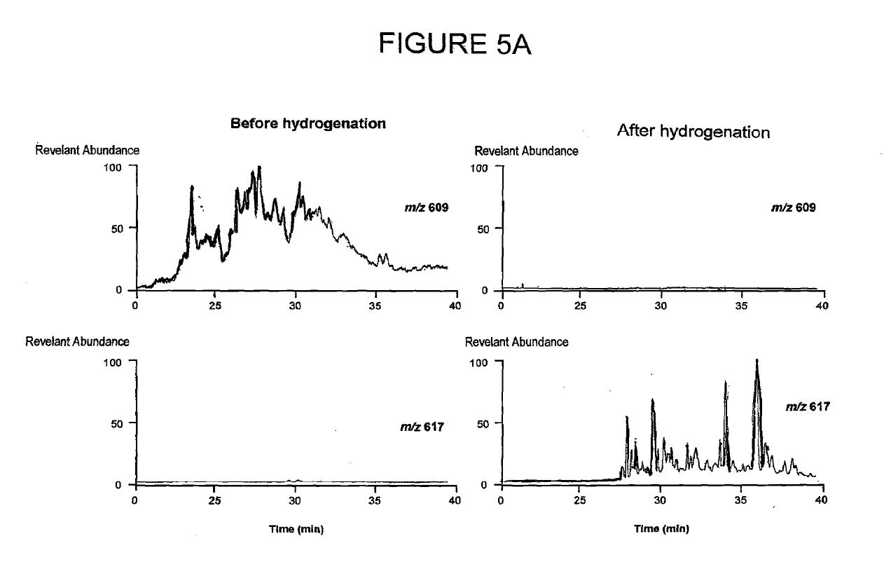

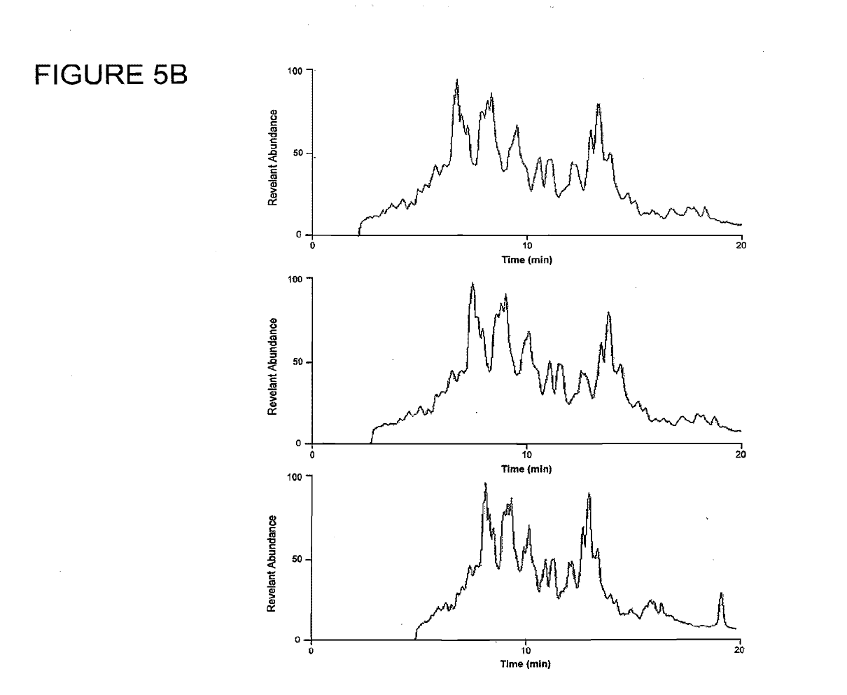

Prior to hydrogenation, no significant peaks are present at m/z 617. However, following hydrogenation, intense peaks appear at m/z 617 while no peaks appear at m/z 609. Thus, catalytic hydrogenation resulted in a mass gain of 8 Da, indicating the presence of four double bonds (FIG. 5A). The presence of epoxide or carbonyl groups was excluded when treatment with HCl or methoxyamine HCl failed to alter the mass chromatogram (FIG. 5B).

Based on the mechanisms postulated for formation of the iFs (Fessel et al., 2002, Proc Natl Acad Sci USA. 99(26):16713-8), two plausible mechanisms for the formation of nFs are proposed: the cyclic peroxide cleavage pathway and the epoxide hydrolysis pathway (FIGS. 6 and 7, respectively). These two mechanisms together predict the formation of 16 distinct regioisomers, each comprised of 32 racemic diastereomers for a total of 512 compounds. The epoxide hydrolysis pathway is predicted to contribute to the formation of all 16 regioisomers, whereas the cyclic peroxide cleavage pathway is predicted to contribute to the formation of only eight of the sixteen regioisomers. The nomenclature “-epox” or “-both” are used in these pathways to be consistent with iFs (Fessel et al., 2002, supra).

### Experimental Example 2

### Analysis of the Unknown Compounds by MS

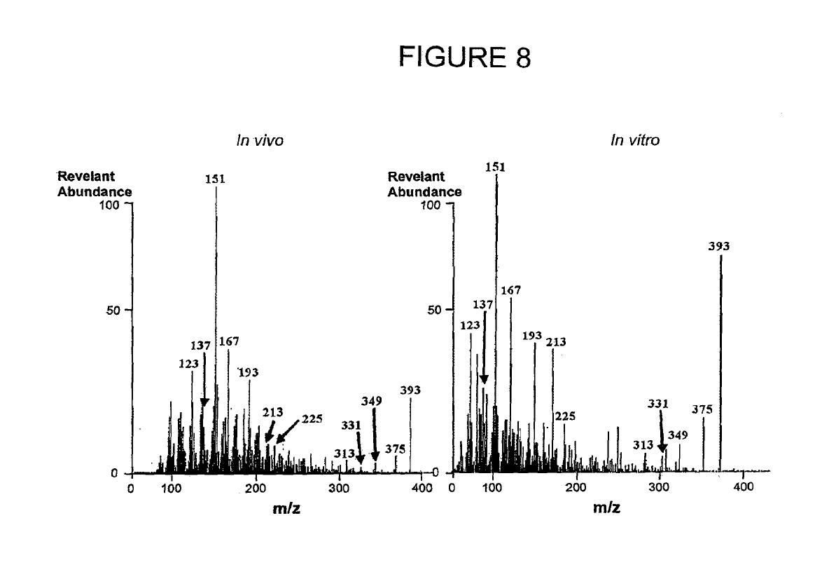

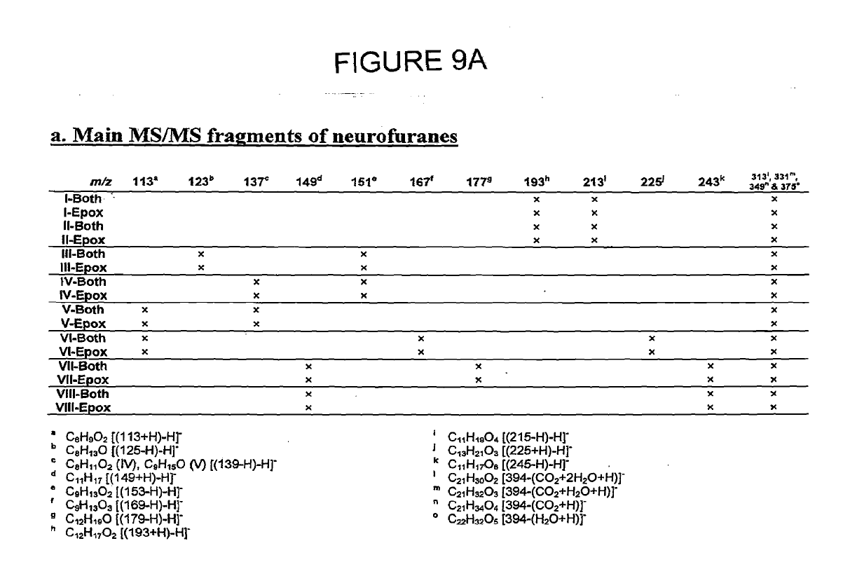

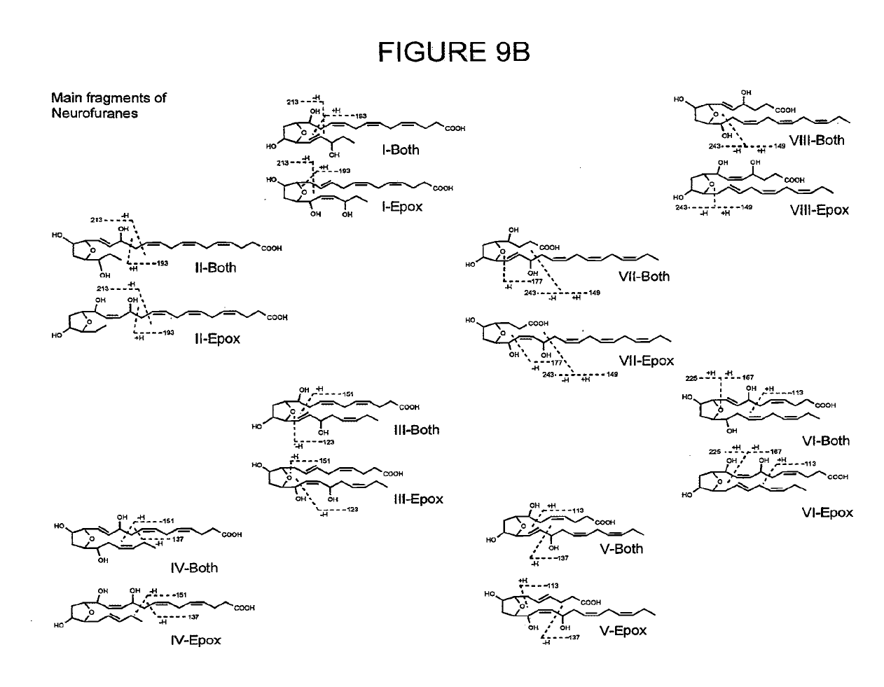

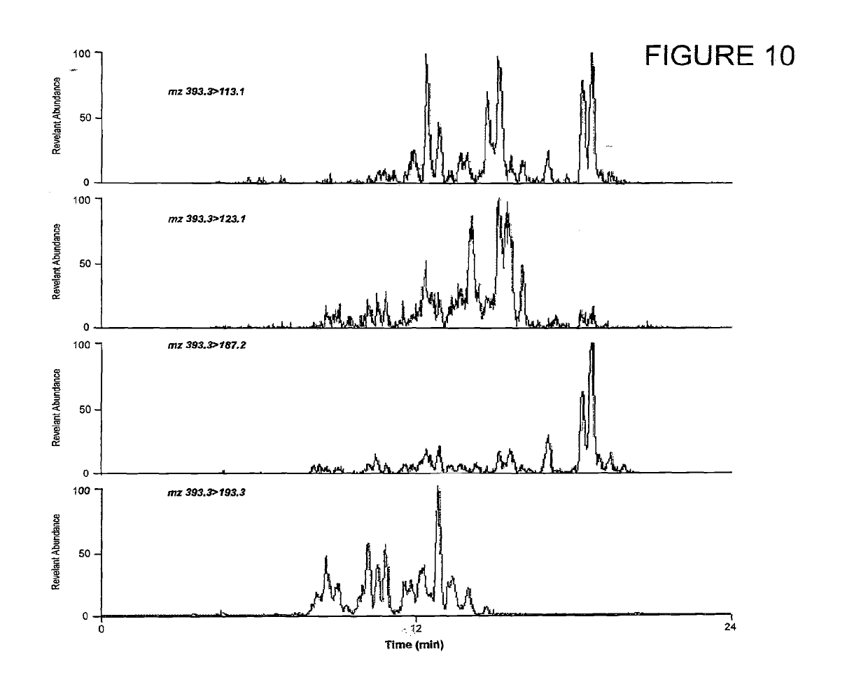

The compounds purified from both in vivo and in vitro oxidation experiments were analyzed by infusion into an electrospray MS/MS, while scanning product ions of m/z 393. Spectra averaged over a period of approximately 9 minutes during the infusion are shown in FIG. 8. The spectra obtained in vivo and in vitro are strikingly similar, differing mainly in the relative abundance of the various product ions. A prominent ion is present at m/z 393, representing the unfragmented precursor ion. A set of product ions were predicted from the postulated structures shown in FIG. 9. SRM analysis of some predicted transitions is shown in FIG. 10. Results from an extract of mouse liver after CCl₄ infusion were similar. The transition of m/z 393→193 was the most abundant one, which could come from I-Both, I-Epox, II-Both and II Epox. Again, the unknown compounds revealed a similar pattern of SRM fragments whether derived in vivo or in vitro.

Overall, these data provide strong evidence that these unknown compounds are nFs.

### Experimental Example 3

### Formation of nFs in Vitro

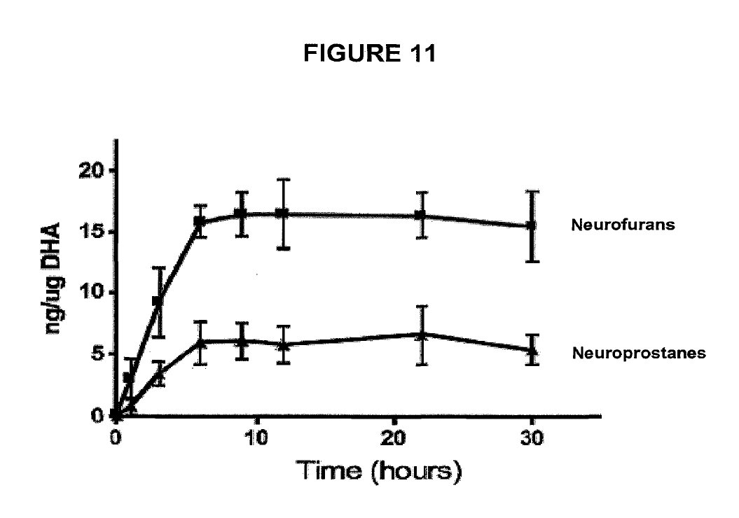

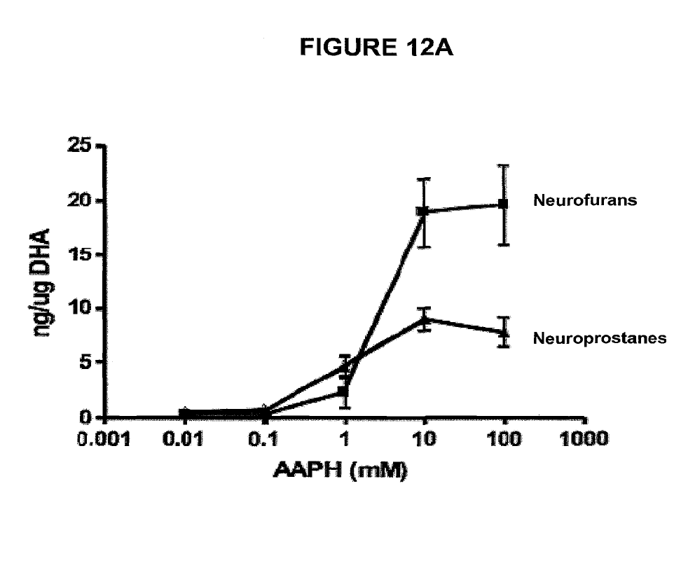

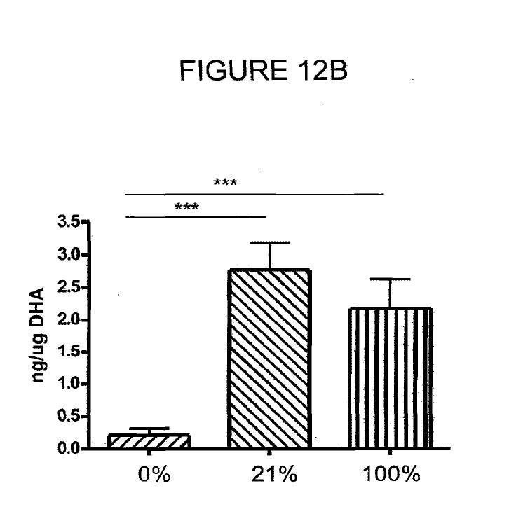

The formation of nFs and nPs by in vitro lipid oxidation reflected both the duration of exposure and the concentration of AAPH. The formation of nFs during oxidation of DHA using AAPH reached a maximum of roughly 15 ng/μg DHA by 6 hours (FIG. 11). nFs were more abundant than the nPs at all time points. This effect was dose related and a maximal response was attained at roughly 10 mM AAPH (FIG. 12A). However, unlike iFs, increasing oxygen tension did not detectably favor nF formation (Fessel et al., 2002, Proc Natl Acad Sci USA. 99(26):16713-8) (FIG. 12B).

### Experimental Example 4

### Formation of nFs in Vivo

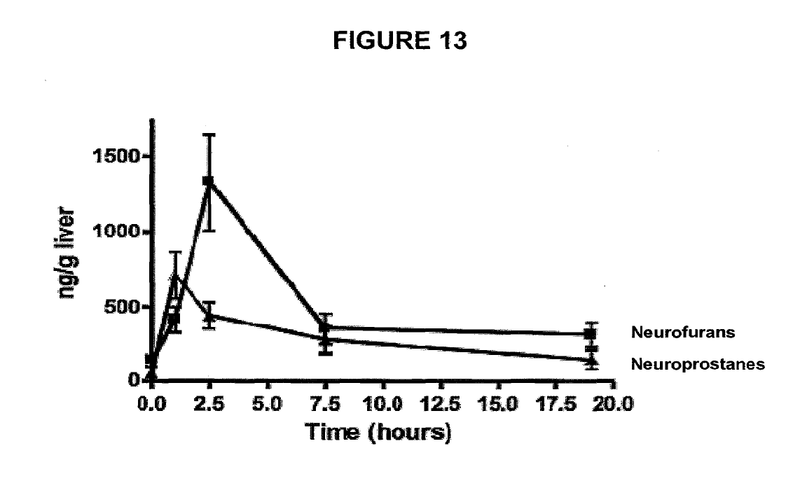

NFs were readily detected in mouse liver. Formation of nFs increased dramatically, from a mean 141.3 ng/g tissue weight before CCl₄ injection to 412.2 ng/g at one hour and to 1330.6 ng/g tissue weight at 2.5 hours after CCl₄ administration. While the nPs also increased dramatically, they were less abundant than the nFs (FIG. 13). Unmetabolized nFs were below the detectable level in mouse plasma and urine in these experiments.

### Experimental Example 5

### nFs in the Tg2576 Transgenic Mouse Model of AD and p47phox Knockout Mice

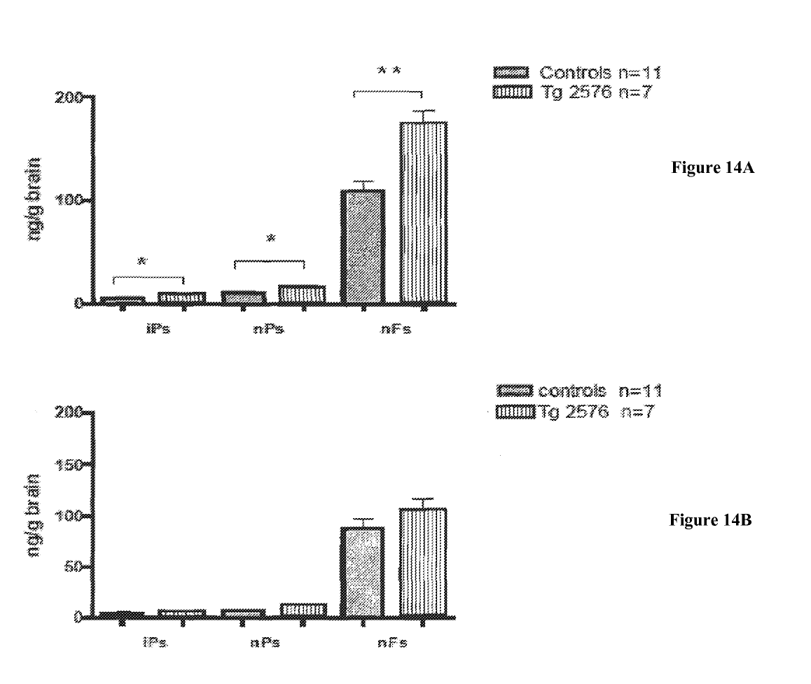

The transgenic mouse line Tg2576 expressing the human amyloid precursor protein with the “Swedish” double mutation KM670/671NL driven by the hamster prior protein promoter (Hsiao et al., 1996, Science 274: 99-102) has been extensively characterized and is a widely used model of AD. These mice develop abundant extracellular amyloid deposits after 12-15 months (Hsiao et al., 1996, Science 274: 99-102; Flood et al., 2002, Neurobiol Aging 23: 335-348). Levels of iPs, nPs and nFs were significantly (p<0.05) elevated in Tg2576 mouse brain cortex (7.1 vs 5.4 ng/g; 14.3 vs 10.5 ng/g and 173.2 vs 109.1 ng/g tissue weight, respectively compared to controls) (FIG. 14A). In contrast, no significant difference was noted in levels of iPs, nPs and nFs in the cerebellum of Tg 2576 vs control mice (FIG. 14B). The cerebellum is an area spared amyloid pathology.

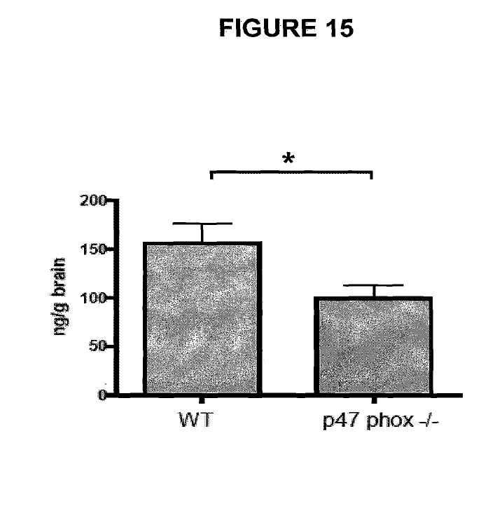

Deletion of p47phox, an essential component of the phagocyte NADPH oxidase (phox), renders murine microglial cells unable to produce superoxide (Lavigne et al., 2001, 15(2):285-7. Epub 2000 Dec. 8). The levels of nFs were significantly (p<0.05) reduced in the p47phox knockout mouse brain cortex from a mean of 156.2 ng/g to 99.3 ng/g tissue weight (FIG. 15).

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1-33. (canceled)

34. A kit for detecting a neurofuran in a sample, the kit comprising a neurofuran standard and an instructional material, wherein the neurofuran standard comprises a substantially purified neurofuran, and wherein the instructional material comprises instructions on detecting the neurofuran in the sample.

35. The kit of claim 34, further comprising a solution for extracting the neurofuran from a biological sample.

36. The kit of claim 35, wherein the biological sample comprises brain tissue.

37. The kit of claim 35, wherein the biological sample comprises cerebrospinal fluid (CSF).

38. The kit of claim 34, wherein the neurofuran is characterized by:
a. having molecular formula of C₂₂H₃₄O₆;
b. presence of a substituted tetrahydrofuran ring
c. presence of three hydroxyl groups;
d. presence of four double bonds;
e. absence of epoxide groups; and
f. absence of carbonyl groups.

39. The kit of claim 38, wherein the neurofuran is further characterized by:
g. eluting at m/z 393 in liquid chromatography/mass spectrometry (LC/MS) analysis; and
h. eluting as m/z 609 in gas chromatography/electron capture/negative ionization/mass spectrometry (GC/EC/NI/MS) analysis after pentafluorobenzyl (PFB) ester and trimethylsilyl (TMS) ether derivatization.

40. The kit of claim 34, further comprising an antibody that specifically binds to the neurofuran.

41. The kit of claim 35, further comprising a biological sample container for carrying the tissue or body fluid sample from a mammal.

42. The kit of claim 41, wherein the tissue sample comprises brain tissue.

43. The kit of claim 41, wherein the body fluid comprises cerebrospinal fluid (CSF).

44. A kit for diagnosing Alzheimer's disease in a mammal, the kit comprising:
a) a solution for use in extracting a neurofuran from a tissue or body fluid sample obtained from the mammal;
b) a negative control solution of the neurofuran, wherein the concentration of the neurofuran is about the same as that in a tissue or body fluid sample of a mammal which is not afflicted with Alzheimer's disease;
c) a positive control solution of the neurofuran, wherein the concentration of the neurofuran is about the same as that in a tissue or body fluid sample of a mammal which is afflicted with Alzheimer's disease;
d) an antibody directed against the neurofuran; and
e) an instructional material comprising instructions how to detect the neurofuran in the tissue or body fluid.

45. The kit of claim 44, further comprising f) a sample container for carrying the tissue or body fluid sample from the mammal

46. The kit of claim 44, wherein the tissue comprises a brain tissue.

47. The kit of claim 44, wherein the body fluid sample comprises cerebrospinal fluid (CSF).

48. The kit of claim 44, wherein the mammal is a human.
